SEQUENCE LISTING (11 sequences)

SEQ ID NO: 1 - DNA, 30 bp, Artificial
  Amplifying primer 1
agggggccgaatactggggcccggattacg

SEQ ID NO: 2 - DNA, 20 bp, Artificial
  Amplifying primer 2
ctcgaagaccgggccatgga

SEQ ID NO: 3 - DNA, 32 bp, Artificial Sequence
  Primer F1
  misc_feature (21)..(22): “n” on pos. 21 and 22 stands for unknown nucleic acid
accgcctgatgaacttcctcnnsgtcgcgtcg

SEQ ID NO: 4 - DNA, 20 bp, Artificial Sequence
  Primer F2
atgtgtggaattgtgagcgg

SEQ ID NO: 5 - DNA, 34 bp, Artificial Sequence
  Primer B1
  misc_feature (12)..(13): “n” on pos. 12 and 13 stands for unknown nucleic acid
gcgcctcgtasnnggtgaagtagtccgtcgtcca

SEQ ID NO: 6 - DNA, 57 bp, Artificial Sequence
  Primer B2
  misc_feature (13)..(14): “n” on pos. 13 and 14 stands for unknown nucleic acid
  misc_feature (46)..(47): “n” on pos. 46 and 47 stands for unknown nucleic acid
ccgttgacggtsnnggtgatgcccatccgctggttgaaggcgaasnngatgaccggc

SEQ ID NO: 7 - DNA, 18 bp, Artificial Sequence
  Primer B3
gattggtgacccgcggca

SEQ ID NO: 8 - DNA, 2163 bp, Pseudomonas SY-77
  misc_feature (1)..(2163): glutaryl amidase
atgctgagagttctgcaccgggcggcgtccgccttggttatggcgactgtgatcggccttgcgcccgccgtcgcctttgcgctggccgagccgacctcgacgccgcaggcgccgattgcggcctataaaccgagaagcaatgagatcctgtgggacggctacggcgtcccgcacatctacggcgtcgacgcgccctcagccttctacggctatggctgggcccaggcgcgcagccacggcgacaatatcctgcgcctgtatggagaagcgcggggcaagggggccgaatactggggcccggattacgaacagacgaccgtctggctgctgaccaacggcgtgccggagcgcgctcagcagtggtatgcgcagcagtcgcctgatttccgcgccaacctcgacgccttcgcggcgggcatcaacgcctatgcgcagcagaaccccgacgacatctcgcccgaggtgcggcaggtgctgccggtttccggcgccgacgtggtggcccacgcccaccgcctgatgaacttcctctatgtcgcgtcgcccggccgcaccctgggcgagggcgacccgccggacctggccgatcaaggatccaactcctgggcggtggcgccgggaaagacggcgaacgggaacgccctgctgctgcagaacccgcacctgtcctggacgacggactacttcacctactacgaggcgcatctcgtcacgccggacttcgaaatctatggcgcgacccagatcggcctgccggtcatccgcttcgccttcaaccagcggatgggcatcaccaataccgtcaacggcatggtgggggccaccaactatcggctgacgcttcaggacggcggctatctgtatgacggtcaggtgcggccgttcgagcggcctcaggcctcgtatcgcctgcgtcaggcggacgggacgacggtcgacaagccgttggagatccgctccagcgtccatggcccggtcttcgagcgcgcggacggcacggccgtcgccgttcgggtcgccggtctggaccggccgggcatgctcgagcagtatttcgacatgatcacggcggacagcttcgacgactacgaagccgctttggcgcggatgcaggtgccgaccttcaacatcgtctacgccgaccgcgaagggaccatcaactacagcttcaacggcgtggcgcccaaacgggccgagggcgacatcgccttctggcaggggctcgtgccgggcgattcctcgcgttacctgtggaccgagacacacccgctggacgatctgccgcgcgtcaccaatccgccgggcggcttcgtgcagaactccaatgatccgccgtggacgccgacctggcccgtcacctacacgcccaaggacttcccctcctatctggcgccccagacgccgcattccctgcgtgcgcaacaaagcgtgcgtctgatgtccgagaacgacgacctgacgctggagcgcttcatggcgctgcagttgagccatcgcgccgtcatggccgaccgcaccttgccggacctgatcccggccgccctgatcgaccccgatcccgaggtccaggcggcggcgcgcctgctggcggcgtgggatcgcgagttcaccagcgacagccgcgccgccctgctgttcgaggaatgggcgcgtctgttcgccggccagaatttcgcaggccaggccggcttcgccacgccctggtcgctggataagccggtcagcacgccttacggcgtccgcgaccccaaggccgccgtcgatcaactgcggaccgccatcgccaacaccaagcgcaaatacggcgcgatcgaccggccgttcggcgacgcctcgcgcatgatcctgaacgacgtgaatgttccgggcgccgccggctacggcaacctgggttccttccgggtcttcacctggtccgatcctgacgaaaacggggttcgcacgcccgtccacggcgagacgtgggtggcgatgatcgagttctccacgccggtgcgggcctatggcctgatgagctacggcaactctcgccagccgggcacgacgcactacagcgatcagatcgaacgcgtgtcgcgcgccgacttccgcgaactgttgctgcggcgagagcaggtcgaggccgccgtccaggaacgcacgcccttcaacttcaagccatga

SEQ ID NO: 9 - protein, 720 aa, Pseudomonas SY-77
  SITE (1)..(720): glutaryl amidase propeptide
MLRVLHRAASALVMATVIGLAPAVAFALAEPTSTPQAPIAAYKPRSNEILWDGYGVPHIYGVDAPSAFYGYGWAQARSHGDNILRLYGEARGKGAEYWGPDYEQTTVWLLTNGVPERAQQWYAQQSPDFRANLDAFAAGINAYAQQNPDDISPEVRQVLPVSGADVVAHAHRLMNFLYVASPGRTLGEGDPPDLADQGSNSWAVAPGKTANGNALLLQNPHLSWTTDYFTYYEAHLVTPDFEIYGATQIGLPVIRFAFNQRMGITNTVNGMVGATNYRLTLQDGGYLYDGQVRPFERPQASYRLRQADGTTVDKPLEIRSSVHGPVFERADGTAVAVRVAGLDRPGMLEQYFDMITADSFDDYEAALARMQVPTFNIVYADREGTINYSFNGVAPKRAEGDIAFWQGLVPGDSSRYLWTETHPLDDLPRVTNPPGGFVQNSNDPPWTPTWPVTYTPKDFPSYLAPQTPHSLRAQQSVRLMSENDDLTLERFMALQLSHRAVMADRTLPDLIPAALIDPDPEVQAAARLLAAWDREFTSDSRAALLFEEWARLFAGQNFAGQAGFATPWSLDKPVSTPYGVRDPKAAVDQLRTAIANTKRKYGAIDRPFGDASRMILNDVNVPGAAGYGNLGSFRVFTWSDPDENGVRTPVHGETWVAMIEFSTPVRAYGLMSYGNSRQPGTTHYSDQIERVSRADFRELLLRREQVEAAVQERTPFNFKP

SEQ ID NO: 10 - protein, 161 aa, Pseudomonas SY-77
  SITE (1)..(161): glutaryl amidase A chain (pos. 28 - 188)
LAEPTSTPQAPIAAYKPRSNEILWDGYGVPHIYGVDAPSAFYGYGWAQARSHGDNILRLYGEARGKGAEYWGPDYEQTTVWLLTNGVPERAQQWYAQQSPDFRANLDAFAAGINAYAQQNPDDISPEVRQVLPVSGADVVAHAHRLMNFLYVASPGRTLGE

SEQ ID NO: 11 - protein, 522 aa, Pseudomonas SY-77
  SITE (1)..(522): glutaryl amidase B chain (pos. 199 - 720)
SNSWAVAPGKTANGNALLLQNPHLSWTTDYFTYYEAHLVTPDFEIYGATQIGLPVIRFAFNQRMGITNTVNGMVGATNYRLTLQDGGYLYDGQVRPFERPQASYRLRQADGTTVDKPLEIRSSVHGPVFERADGTAVAVRVAGLDRPGMLEQYFDMITADSFDDYEAALARMQVPTFNIVYADREGTINYSFNGVAPKRAEGDIAFWQGLVPGDSSRYLWTETHPLDDLPRVTNPPGGFVQNSNDPPWTPTWPVTYTPKDFPSYLAPQTPHSLRAQQSVRLMSENDDLTLERFMALQLSHRAVMADRTLPDLIPAALIDPDPEVQAAARLLAAWDREFTSDSRAALLFEEWARLFAGQNFAGQAGFATPWSLDKPVSTPYGVRDPKAAVDQLRTAIANTKRKYGAIDRPFGDASRMILNDVNVPGAAGYGNLGSFRVFTWSDPDENGVRTPVHGETWVAMIEFSTPVRAYGLMSYGNSRQPGTTHYSDQIERVSRADFRELLLRREQVEAAVQERTPFNFKP

(19) United States
(12) Patent Application Publication
(10) Pub. No.: US 2006/0292665 A1
(43) Pub. Date: Dec. 28, 2006

(54) Glutaryl amidases and their uses

(75) Inventors: Charles Fredrik Sio, Eindhoven (NL); Ludwina Gerharda Otten, Delft (NL); Robbert Hans Cool, Groningen (NL); Wilhelmus Johannes Quax, Kropswolde (NL)

Correspondence Address: TRASK BRITT, P.O. BOX 2550, SALT LAKE CITY, UT, 84110, US

(73) Assignee: Stichting voor de Technische Wetenschappen, Utrecht (NL)

(21) Appl. No.: 11/448,141

(22) Filed: Jun. 5, 2006

Related U.S. Application Data
(63) Continuation of application No. PCT/NL04/00850, filed on Dec. 6, 2004.

(30) Foreign Application Priority Data
Oct. 20, 2004 (EP) 04077894.6
May 7, 2004 (EP) 04076377.3
Dec. 5, 2003 (EP) 03078861.6

(51) Int. Cl. C12P 35/02; C12P 21/06; C07H 21/04; C07D 501/00; C12N 9/80; C07D 501/14; C12N 15/74; C12N 1/21

(52) U.S. Cl. 435/51; 435/69.1; 435/228; 435/252.34; 435/471; 536/23.2; 540/222

(57) ABSTRACT

The invention relates to a new class of isolated, single or multiple mutated glutaryl amidases from Pseudomonas SY-77, or a functional part, derivative or analogue thereof. The invention also relates to a method for preparing 7-aminocephalosporanic acid from cephalosporin C, using a glutaryl amidase according to the invention. The invention also provides a method for preparing aminodesacetoxycephalosporanic acid.

### CROSS-REFERENCE TO RELATED APPLICATIONS

This application is a continuation of PCT International Patent Application No. PCT/NL2004/000850, filed on Dec. 6, 2004, designating the United States of America, and published, in English, as PCT International Publication No. WO 2005/054452 A2 on Jun. 16, 2005, which application claims priority to European Patent Application Serial No. 04077894.6, filed on Oct. 20, 2004, European Patent Application Serial No. 04076377.3, filed on May 7, 2004, and European Patent Application Serial No. 03078861.6, filed on Dec. 5, 2003, the contents of the entirety of each of which are hereby incorporated herein by this reference.

### TECHNICAL FIELD

The present invention relates to a new class of glutaryl amidases and variants and their uses, in particular, their use in a process for the preparation of 7-aminocephalosporanic acid (7-ACA).

### BACKGROUND

It is known that 7-ACA can be used as the starting product for the semi-synthetic preparation of various cephalosporin antibiotics and that there exist various processes to prepare 7-ACA on a large scale. Among these known processes, the enzymatic processes are preferred, from both economic and environmental perspective.

In one of the known production processes, cephalosporin C is decarboxylated by chemical oxidation to form glutaryl-7-ACA, and the so obtained substrate is subsequently processed to form 7-ACA using the enzyme glutaryl amidase. This process has, however, the disadvantage that it involves a high level of environmental pollution, resulting in high costs.

In another known production process (as, for example, described in WO03076640), cephalosporin C is firstly subjected to an oxidative decarboxylation treatment that is carried out enzymatically with the help of D-amino acid oxidase. The glutaryl-7-ACA so obtained is then converted into 7-ACA using the enzyme glutaryl amidase. Although the latter enzymatic process is already much more attractive than the former, it still involves several process steps and, therefore, there is still significant room to develop an improved process for the conversion of cephalosporin C into 7-ACA.

In view of the commercial interest of such an application, many attempts have been made to isolate an amidase that can hydrolyze cephalosporin C to produce 7-ACA. Enzymes from different organisms have been isolated and tested on their capability to hydrolyze cephalosporin C. Examples can be found in patents WO0224879, U.S. Pat. Nos. 5,229,274 and 4,774,179, EP-A-0283218, and EP-A-0322032. Nevertheless, so far, the cephalosporin C hydrolysis activity in any of the known amidases is too low for commercial interest.

Attempts have also been made to mutate glutaryl amidase at specific positions in order to increase the cephalosporin C hydrolysis activity. For example, the glutaryl amidase as described in U.S. Pat. No. 4,774,179 was mutated at several positions, but so far, no commercially usable variants have been obtained [ref. 1-4]. Further in WO 02/072806, mutations in another glutaryl amidase have been proposed on the basis of structural modeling without any experimental substantiation [ref. 5]. In yet another approach, mutations were introduced in a random manner, after which, variants with an activity towards a desired substrate were selected. As an example of the latter, the glutaryl amidase of Pseudomonas SY-77 was mutated and variants of the amidase with the desired activity were isolated by growth on selective medium. Several mutants, amongst which a variant carrying the mutation Tyr178 into His, were claimed (U.S. Pat. No. 5,457,032). The latter mutant was shown to have increased hydrolysis activity on adipyl-7-aminodesacetoxycephalosporanic acid (7-ADCA; ref. 6). This compound is, like 7-ACA, an industrially important reagent for the synthesis of semi-synthetic cephalosporin antibiotics [ref. 7]. However, in spite of the fact that this mutant was claimed more than ten years ago, no activity for cephalosporin C was demonstrated so far.

It is further observed that mutations at other positions in Pseudomonas diminuta cephalosporin acylase were predicted by modeling studies and claimed to increase the cephalosporin C hydrolysis activity of a glutaryl amidase several fold [ref. 8]. However, no formation of 7-ACA was shown. Only a few variants were predicted and there was clearly no teaching for further improvement by making, for instance, combinations of different mutations.

In order to isolate new mutants of Pseudomonas SY-77 with increased hydrolysis activity towards adipyl-7-ADCA, an unbiased approach using mutagenesis and growth selection on solid medium containing adipyl-leucine was used recently [ref. 6, 9]. However, no activity on cephalosporin C was shown.

Surprisingly, it has now been found that a very attractive single-enzyme process can be developed for the production of 7-ACA from cephalosporin C when use is made of a newly developed class of glutaryl amidases.

### DISCLOSURE OF THE INVENTION

Accordingly, one aspect of the present invention relates to an isolated or single mutated glutaryl amidase from Pseudomonas SY-77, or a functional part, derivative or analogue thereof, having an amino acid at position 266 other than asparagine, histidine or serine.

With the term “single mutated” is meant that the glutaryl amidase is derived from a naturally occurring glutaryl amidase that has only been mutated in one position. In other words, Asn266 has been mutated into an amino acid other than asparagine.

A major advantage of this newly developed class of glutaryl amidases is that they enable the direct conversion of cephalosporin C into 7-ACA in a much simpler and cheaper way. Further, these variants can suitably be used to prepare 7-ACA on an industrial scale. In such processes, the glutaryl amidase according to the present invention binds cephalosporin C as a substrate and catalyzes the conversion of the cephalosporin C into 7-ACA, whereby the amino acid at position 266 directly or indirectly increases the cephalosporin C hydrolysis activity of the amidase.

Preferably, the present invention relates to an isolated or single mutated glutaryl amidase from Pseudomonas SY-77, or a functional part, derivative or analogue thereof, having a glutamine, methionine or tryptophan at position 266. More preferably, such glutaryl amidase has a glutamine or methionine at position 266. Most preferably, such glutaryl amidase has a glutamine at position 266.

Another aspect of the present invention is that the effect of a mutation in position 266 on cephalosporin C hydrolysis activity can be improved by the additional presence of other mutations in or near to the substrate binding pocket. Amino acids that directly or indirectly contribute to substrate binding, or that may otherwise contribute to substrate specificity, are found in positions: Leu177, Tyr178, Val179, His221, Leu222, Phe229, Tyr231, Gln248, Arg255, Phe256, Thr267, Val268, Met271, Gln291, Met347, Tyr351, Thr374, Phe375 and/or Asn442. It cannot be excluded that variants of glutaryl amidase with mutations in these positions have a high cephalosporin C hydrolysis activity even in combination with Asn266.

Accordingly, another attractive aspect of the invention relates to an isolated or multiple mutated glutaryl amidase from Pseudomonas SY-77, or a functional part, derivative or analogue thereof, having an amino acid other than its native amino acid at position Asn266, and having an amino acid other than its native amino acid at one or more of the following positions: Leu177, Tyr178, Val179, His221, Leu222, Phe229, Tyr231, Gln248, Arg255, Phe256, Thr267, Val268, Met271, Gln291, Met347, Tyr351, Thr374, Phe375 and/or Asn442.

With the term “multiple mutated” is meant that the glutaryl amidase is derived from a naturally occurring glutaryl amidase that has been mutated in at least two positions. In other words, Asn266 has been mutated into another amino acid, whereas in addition, one or more other native amino acids have been mutated into other amino acids.

As mentioned hereinabove, these glutaryl amidases display an improved efficiency in the preparation of 7-ACA from cephalosporin C.

Preferably, the isolated or multiple mutated glutaryl amidase according to the invention has, in addition to an amino acid other than asparagine at position 266, one or more mutations at the following positions: Tyr178, Leu222, Tyr231, Gln248, Arg255, Val268, Tyr351 and/or Phe375.

Most preferably, the isolated or multiple mutated glutaryl amidase comprises, in addition to an amino acid other than asparagine at position 266, one or more mutations at the following positions:

Tyr178,

Tyr231,

Arg255,

Phe375,

Tyr178+Tyr231,

Tyr178+Arg255,

Tyr178+Phe375,

Tyr231+Arg255,

Tyr231+Phe375,

Tyr231+Arg255+Phe375,

Arg255+Phe375,

Tyr178+Tyr231+Arg255,

Tyr178+Tyr231+Phe375,

Tyr178+Arg255+Phe375, or

Tyr178+Tyr231+Arg255+Phe375.

All amino acid numbering in the present application is, with respect to the propeptide chain, as shown in FIG. 1. The numbering applies to equivalent positions in amino acid sequences of other glutaryl amidases as determined, for example, by sequence alignment.

In the context of the present invention, a mutation of a position in the glutaryl amidase implies the incorporation of any of the 20 natural amino acids other than the native amino acids at that particular position in the glutaryl amidase (as listed in FIG. 1), or the incorporation of a non-natural amino acid at that particular position or a modification of the amino acid at that particular position.

The glutaryl amidase is obtained from a Pseudomonas SY-77, or is a functional part, derivative or analogue thereof. In the context of the present application, derivates or analogues of Pseudomonas SY-77 glutaryl amidase include: Pseudomonas C427 glutaryl amidase [ref. 10], Pseudomonas sp. 130 glutaryl amidase [ref. 11, 12], Pseudomonas GK-16 glutaryl amidase [ref. 13, 14], and Pseudomonas diminuta KAC-1 glutaryl amidase [ref. 15, 16].

The glutaryl amidase of Pseudomonas SY-77 has, for instance, been described in the references [ref. 6, 17]. The homologues of Pseudomonas SY-77 glutaryl amidase as used in accordance of the present invention have, for instance, been described in the references [ref. 10-16].

The present invention also provides a polynucleotide encoding a variant of a glutaryl amidase as described hereinabove.

The present invention further provides a nucleic acid vector comprising a polynucleotide encoding a variant of a glutaryl amidase as described hereinabove.

Additionally, the present invention provides a host cell comprising a polynucleotide encoding a variant of a glutaryl amidase as described hereinabove.

The present invention also relates to a method for producing any one of the glutaryl amidases according to the present invention, which method comprises culturing the hereinabove-mentioned host cell under suitable conditions that allow for expression of the glutaryl amidase in the host cell.

In the context of the invention, variants of the glutaryl amidase will be produced in host cells by expression of the encoding gene or by production in a cell-free system. Host cells principally can be prokaryotic or eukaryotic cells, but are by preference prokaryotic. More preferably, the host cells are bacterial cells such as Escherichia coli or Bacillus subtilis or Pseudomonas. Examples of such expression systems can be found in ref. 6 and 18. A cell-free system, such as the Rapid Translation System (Roche), can also be used to produce the variants. Alternatively, both subunits of the glutaryl amidase can be co-expressed as, for example, described in ref. 11.

By way of an example, recombinant E. coli clones expressing the variant of a glutaryl amidase under control of a strong promoter according to the present invention is typically cultured to a 0.1-10 liter scale. The cells are initially concentrated by centrifugation. Subsequently, they are resuspended in a small volume of a suitable buffer and disrupted. After centrifugation to remove the cell debris, the supernatant is used in a purification strategy to obtain the purified enzyme.

By way of an example, and more suitable for smaller quantities, the cells may be lysed by sonication, followed by a three-step purification using different chromatographic steps as described hereinbelow in Example 1.

Alternatively, and more suitable for an industrial scale, cells may be resuspended in 100 mM phosphate buffer at a pH of 7.2 and disrupted by means of a French press. To remove the cell debris, the suspension is then centrifuged at 10,000 rpm for 15 minutes and the supernatant is transferred to another tube. The enzyme-containing supernatant solution is then rebuffered in an Amicon ultrafiltration cell or by diafiltration against 0.8-1.5 M, more preferably in the range of from 1-1.5 M, potassium phosphate buffer at a pH of 7.8 at 4° C. with simultaneous concentration. If the concentrated solution is left to stand in cold conditions, the enzyme crystallizes out. The crystals may be enriched by gentle centrifugation and washed with the above-mentioned buffer. In this way, large amounts of purified enzyme can be obtained in a simple, swift and inexpensive manner, without using expensive chromatographic purification steps.

In a preferred embodiment, cephalosporin C hydrolysis is carried out at a pH in the range of from pH 7.5 to 8.5, more preferably in the range of from 7.5 to 8.2. In that way, decomposition of cephalosporin C is minimized.

The concentration of cephalosporin C in the above method lies preferably in the range from 20-200 mM, more preferably in the range of from 50 to 120 mM.

In the above method, the variant of the glutaryl amidase is suitably used in a concentration in the range of from 0.25 to 10 mg/ml in the reaction mixture, and preferably in the range of from 1 to 4 mg/ml.

The above method is suitably carried out at a temperature in the range of from 15 to 45° C., preferably in the range of from 25 to 40° C.

The reaction time for cephalosporin C hydrolysis is suitably in the range of from one to forty hours, preferably in the range of from one to twenty hours.

The variants of a glutaryl amidase according to the present invention can also be applied carrier-fixed. In this way, a further reduction of costs can be established. Suitable carrier materials that can be used for this purpose have, for instance, been described in ref. 19. Alternatively, variants of a glutaryl amidase according to the present invention can also be applied as carrier-free cross-linked enzymes as, for instance, described in Cao et al. [ref. 20].

The variants of a glutaryl amidase according to the present invention can be used to produce 7-ACA directly from cephalosporin C.

Accordingly, the present invention also provides a method for preparing 7-ACA from cephalosporin C, which method comprises reacting cephalosporin C with an isolated, single mutated or multiple mutated glutaryl amidase from Pseudomonas SY-77, or a functional part, derivative or analogue thereof, having an amino acid at position 266 other than asparagine under suitable conditions such that the glutaryl amidase cleaves the cephalosporin C directly to form the 7-aminocephalosporanic acid.

In this method, any of the glutaryl amidases according to the present invention can suitably be used.

Preferably, the present invention relates to a method for preparing 7-ACA from cephalosporin C, which method comprises reacting cephalosporin C with a glutaryl amidase under suitable conditions, such that the variant of a glutaryl amidase cleaves the cephalosporin C directly to form the 7-ACA, wherein the glutaryl amidase comprises an isolated or multiple mutated glutaryl amidase from Pseudomonas SY-77, or a functional part, derivative or analogue thereof, having an amino acid other than its native amino acid at position Asn266, and having an amino acid other than its native amino acid at one or more of the positions Leu177, Tyr178, Val179, His221, Leu222, Phe229, Tyr231, Gln248, Arg255, Phe256, Thr267, Val268, Met271, Gln291, Met347, Tyr351, Thr374, Phe375 and/or Asn442.

Surprisingly, it has also been found that the mutation of Tyr178 into another amino acid in Pseudomonas SY-77 glutaryl amidase increases hydrolysis activity on cephalosporin C (see Table 2). This particular mutant has been isolated (ref. 6) but its unexpectedly high activity on cephalosporin C was not known before.

Accordingly, the present invention also relates to a method for preparing 7-aminocephalosporanic acid from cephalosporin C, which method comprises reacting cephalosporin C with an isolated, single or multiple mutated glutaryl amidase from Pseudomonas SY-77, or a functional part, derivative or analogue thereof, having an amino acid other than its native amino acid at position Tyr178, under suitable conditions such that the glutaryl amidase cleaves the cephalosporin C directly to form the 7-aminocephalosporanic acid.

Preferably, the glutaryl amidase is a single mutated glutaryl amidase.

More preferably, the amino acid at position Tyr178 of the glutaryl amidase is a histidine.

Surprisingly, it has further been found that some of the single mutants that were isolated have a high hydrolysis activity with respect to the production of adipyl-7-aminodesacetoxycephalosporanic acid (7-ADCA). This compound is, like 7-ACA, an industrially important reagent for the synthesis of semi-synthetic antibiotics.

The present invention, therefore, also relates to a method for preparing 7-ADCA, which comprises reacting adipyl-7-ADCA with an isolated or single mutated glutaryl amidase from Pseudomonas SY-77, or a functional part, derivative or analogue thereof, having an amino acid other than its native amino acid at position Asn266, under suitable conditions such that the glutaryl amidase cleaves adipyl-7-ADCA to form the 7-ADCA. Preferably, the amino acid at position 266 of the glutaryl amidase is a glutamine, methionine, phenylalanine, tryptophan or tyrosine. More preferably, the amino acid is a methionine or glutamine.

It will be appreciated by the person skilled in the art that the variants with increased hydrolysis activity to produce 7-ACA or 7-ADCA can also be used for the synthesis of new cephalosporin antibiotics from these compounds [ref. 21]. Hence, the present invention also relates to a method for preparing 7-ACA-based antibiotics comprising incubating 7-ACA in the presence of the glutaryl amidase according to the present invention with alkyl side chains under suitable synthesizing conditions.

The glutaryl amidases according to the present invention consist of two sub-units of different sizes that are formed from a propeptide comprising amino acids through processing steps taking place in the periplasm of the expression organism to form a functional enzyme. The small sub-unit (A chain) comprises 161 amino acids in the functional enzyme, the large sub-unit (B chain) 522 amino acids. The protein sequences of the A and B chains are shown in the sequence listing part of the description respectively. Numbering of residues throughout is with reference to the amino acid sequences. However, it will be understood that where other glutaryl amidases are used, the numbering applies to the functionally corresponding residues in those sequences that do not have exactly the same numbering as in the sequence listing part. Furthermore, it will be understood by a person skilled in the art that functional parts of different sizes of the glutaryl amidase or derivative or analogue can be derived without significantly affecting hydrolysis activity.

Mutations in the glutaryl amidase gene sequence may be introduced, for example, using standard site-directed mutagenesis techniques, such as those described in ref. 22.

As mentioned earlier, the present invention also provides a polynucleotide encoding a variant of a glutaryl amidase according to the present invention. Such polynucleotide may comprise DNA or RNA. The polynucleotide may include synthetic or modified nucleotides. Various methods to modify oligonucleotides are known in the art. These methods include the use of methylphosphonate and phosphorothioate backbones, with addition of acridine or polylysine chains at the 3′ and/or 5′ ends of the molecule. The polynucleotides as described hereinabove may be modified by any method available in the art. It will be appreciated by the skilled person in the art that various different polynucleotides can encode the same polypeptide as a result of the degeneracy of the genetic code.

Another aspect of the invention relates to a method for developing a variant of a cephalosporin amidase with high cephalosporin C hydrolysis activity. In this method, use is made of a particular combination of techniques.

Accordingly, the present invention also relates to a method for developing a glutaryl amidase with cephalosporin C hydrolysis activity comprising the steps of:
1) applying a mutagenesis technique in order to create a large library of mutants of a glutaryl amidase according to the present invention that carries a mutation in one or more positions; and subsequently
2) applying a selection procedure comprising growth of proper host cells that express variants of the glutaryl amidase on solid medium containing a selective, aminoadipyl-containing compound and identification of variants with the desired enzymatic activity.

This process enables the identification and preparation of cephalosporin amidases that display a high cephalosporin C hydrolysis activity.

The present invention will now be shown by means of the following Examples, which are non-limiting and have been included for illustration purposes only.

### BRIEF DESCRIPTION OF THE FIGURES

FIG. 1 depicts the amino acid sequence and corresponding DNA sequence (SEQ ID NO:_) of the propeptide of SEQ ID NO:_, including the A chain (SEQ ID NO:_) and B chain (SEQ ID NO:_).

FIG. 2 is a bar graph depicting results from Example 6.

FIG. 3 is a bar graph depicting results from Example 7.

FIG. 4, in three graphs, graphically depicts the kinetic parameters towards adipyl-7-ADCA and glutaryl-7-ACA of several variants of glutaryl amidase. Values given are mean +/−S.D. of at least three independent measurements. Values for adipyl-7-ADCA are shown in black, while glutaryl-7-ACA is shown in grey.

### DETAILED DESCRIPTION OF THE INVENTION

### EXAMPLES

### Example 1

### Preparation and Purification of Pseudomonas SY-77 Glutaryl Amidase Mutants Modified at Position 266

The megaprimer method [ref 23] was used in order to introduce the 19 amino acids other than asparagine on position 266. First, the plasmid pMcSY-2 comprising the wild-type (WT) sequence for Pseudomonas SY-77 glutaryl amidase and a primer with a randomized codon 266 was used in a PCR reaction. The PCR fragment was digested with restriction endonucleases BglII and SstII, and cloned into vector pMcSY-2 digested with the same endonucleases by ligation. Plasmid DNA of a total of 96 transformants was sequenced. This resulted in the isolation of mutants carrying 17 different amino acids at this position in a single mutagenesis round. The missing three mutants were made by site-directed mutagenesis using specific primers.

Escherichia coli DH10B cells containing the plasmid encoding the (mutant) enzyme were grown in 300 ml 2*YT-medium at 25° C. After 30 hours, the cells were harvested by centrifugation. A cell-free extract was made by sonication (10 minutes, output 4, 50% duty cycle on a Sonifier 250, Branson) and centrifugation (30 minutes at 14,000 rpm). This extract was used to purify the enzyme in a three-step protocol on a Duoflow system (Bio-Rad) using columns from Amersham Pharmacia. The cell-free extract was loaded onto a HiTrapQ column and the protein eluted with a linear gradient of 0-1 M NaCl in 50 mM Tris-HCl pH 8.8. After analysis on SDS-PAGE, the fractions containing the enzyme were loaded onto a HiTrap PhenylSepharose HP column. The protein was eluted with a gradient of 0.7-0 M (NH₄)₂SO₄. The pooled fractions containing the enzyme were desalted on a HiPrep 26/10 Desalting column and reloaded onto a HiTrapQ column. After rinsing with 220 mM NaCl, the protein was eluted with 330 mM NaCl. The pooled fractions were stored at −20° C. Typical yields were 10 mg of more than 90% pure enzyme per liter of culture.

### Example 2

### Conversion of Cephalosporin C into 7-aminocephalosporanic Acid

For the determination of activity on cephalosporin C, 70 μg of various SY-77 glutaryl amidase variants and wild-type enzymes were incubated for five hours at 37° C. with 10 mM cephalosporin C in 300 μl of phosphate buffer (20 M, pH 7.5). Aliquots of 40 μl were taken from the reaction mixture every hour and transferred to 140 μl of 0.5 M acetate buffer pH 4.5. 20 μl of a 1 mg/ml fluorescamine in acetone solution was added, and the absorbance at 380 nm was measured on a spectrophotometer after one hour incubation. The product of the deacylation reaction, 7-aminocephalosporanic acid, forms a complex with fluorescamine that absorbs light of 380 nm wavelength [ref. 24].

The mean slope of the conversion of cephalosporin C in the first four hours was determined and normalized towards WT.

### Example 3

### Construction of a Library of Glutaryl Amidase Mutants Randomized at Positions 266 and 375

A pool of vectors harboring glutaryl amidase genes with 20 different codons encoding all 20 naturally occurring amino acids at position 375 was digested with restriction endonucleases NcoI and HindIII, and the small fragment was ligated into the large fragments of a pool of vectors harboring glutaryl amidase genes with 20 different codons encoding all 20 naturally occurring amino acids at position 266 digested with the same restriction endonucleases. This pool of vectors harboring glutaryl amidase genes with altered codons encoding the amino acids at positions 266 and 375 was transformed to E. coli DH10B cells, resulting in a mutant library in which positions 266 and 375 were completely randomized.

### Example 4

### Construction of a Library of Glutaryl Amidase Mutants Randomized at Positions 178, 231, 255, 266 and 375

A mutant library of glutaryl amidase genes, which are randomized in five codons (encoding amino acids at positions 178, 231, 255, 266 and 375), was made by the megaprimer method as described by Landt et al. [ref. 23]. Primers according to parts of the glutaryl amidase gene, but with randomized codons encoding amino acids at position 178, 231, and 255+266, were used to amplify the section containing the codons encoding these four positions of the glutaryl amidase gene. The nucleotide sequences of these primers are listed in Table 1. The library was constructed in four steps:

| | Forward Primer | Backward Primer | Product |
|---|---|---|---|
| Step 1. | F1 | B1 | P1 |
| Step 2. | F2 | P1 | P2 |
| Step 3. | P2 | B2 | P3 |
| Step 4. | P3 | B3 | P4 |

This product P4 was digested with EcoO190I and NcoI, and ligated into the large fragments of a pool of vectors harboring glutaryl amidase genes with 20 different codons encoding all 20 amino acids at position 375 digested with the same restriction endonucleases. This pool of vectors harboring glutaryl amidase genes with altered amino acids at positions 178, 231, 255, 266 and 375 was transformed to E. coli DH10B cells, resulting in a mutant library in which positions 178, 231, 255, 266 and 375 were completely randomized.

### Example 5

### Selection of Variants of Pseudomonas SY-77 on Hydrolysis Activity for Aminoadipyl-leucine or other Aminoadipyl-amino Acids

A library as described in Example 4 was selected on minimal medium containing aminoadipyl-leucine as sole leucine source. 10⁷ different colonies were starved by incubation in 0.9% NaCl for two hours at 37° C. and plated onto selective minimal medium plates containing 0.1 mg/ml aminoadipyl-leucine as a sole leucine source. Approximately 10,000 viable E. coli DH10B cells containing the mutant plasmids were spread onto each plate. Plates were incubated at 30° C. for six days. Every day, newly appearing colonies were marked. Colonies were selected on the basis of their date of appearance and size. Fifty-seven colonies were picked and grown overnight at 30° C. From each culture, the plasmid was isolated and the presence of glutaryl amidase was detected using a polyclonal antibody against the glutaryl amidase of Pseudomonas SY-77. Plasmid were retransformed to E. coli DH10B cells and streaked again on minimal medium containing adipyl-leucine as sole leucine source to ensure unique colonies were obtained. Single colonies from these second plates should be used to inoculate an overnight culture to determine acylase activity and to isolate plasmid DNA for sequence determination.

### Example 6

### Some Single Mutated Variants of Pseudomonas SY-77 Glutaryl Amidase with Increased Cephalosporin C Hydrolysis Activity

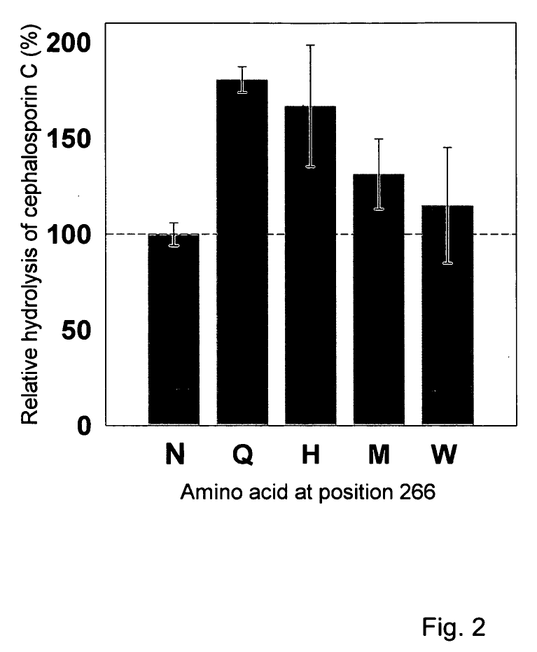

Wild-type (WT) and 15 glutaryl amidase variants mutated at position 266 were produced in E. coli DH10B and purified up to 95% purity. The four glutaryl amidases comprising single mutations N266R, N266I, N266K and N266V could not be produced. Twenty μg of the mutant and WT enzymes were incubated at 37° C. with 10 mM cephalosporin C. The WT enzyme and the variants carrying mutants Asn266 into Gln, Asn266 into His, Asn266 into Met or Asn266 into Trp showed some hydrolysis of the cephalosporin C after 20 hours. These activities were too low to be able to determine Kₘ and k_{cat}. In order to avoid long incubation times that may lead to enzyme destabilization, the conversion rate of the different mutants was calculated from an assay with more enzyme (70 μg) as given in Example 2. The mean slope of the conversion of cephalosporin C in the first four hours was determined and normalized towards WT. All the tested variants, carrying mutants Asn266 into Gln, Asn266 into His, Asn266 into Met or Asn266 into Trp, showed an improved conversion of CPC compared to the activity of the wild-type enzyme. Variants carrying mutations Asn266 into Gln or Asn266 into His displayed almost a two-fold improved conversion of cephalosporin C over wild-type, whereas the variant carrying mutation Asn266 into Met showed an improvement of about 30% (FIG. 2). Hence, this example clearly shows that the variants according to the present invention are indeed very effective enzymes for the conversion of cephalosporin C into 7-ACA.

Similarly, the variant carrying mutation Tyr178 into His was shown to be three-fold more active towards cephalosporin C (Table 2).

### Example 7

### Some Multiple Mutated Variants of Pseudomonas SY-77 Glutaryl Amidase with Increased Cephalosporin C Hydrolysis Activity

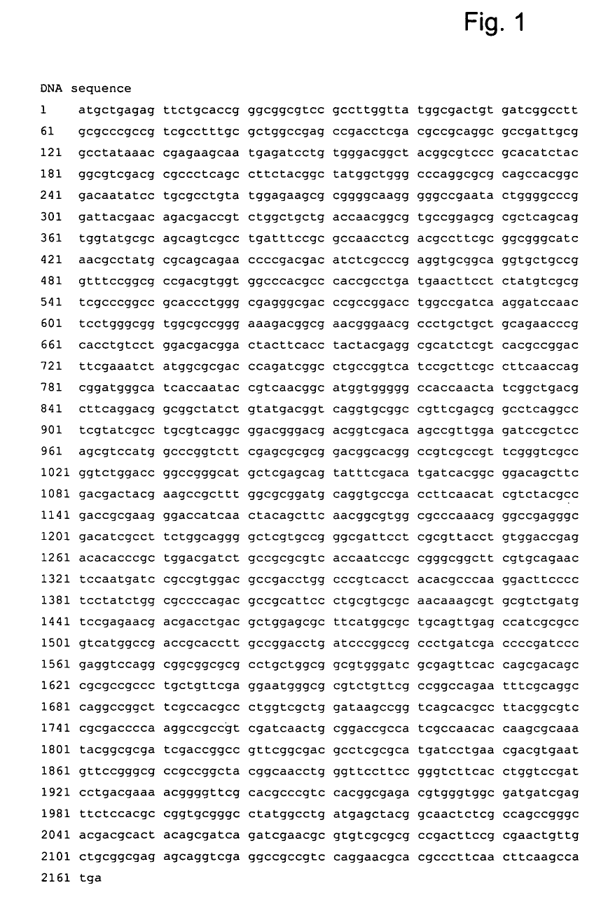

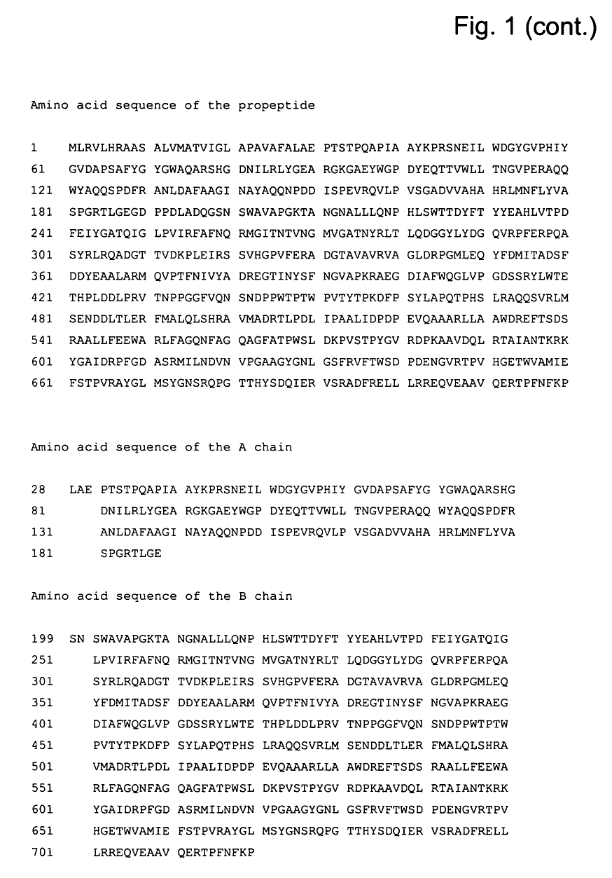

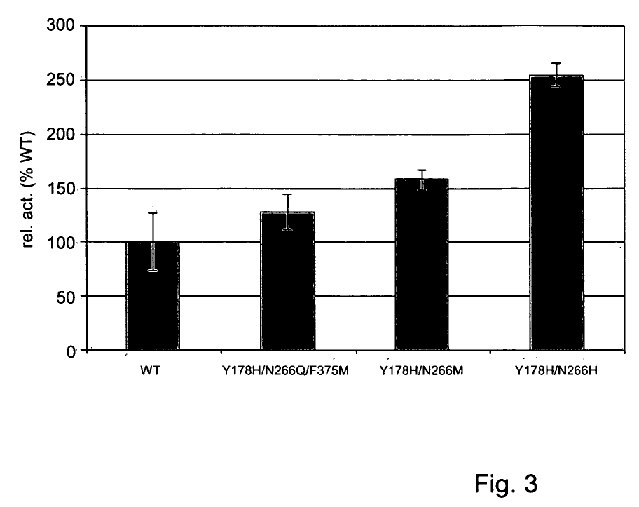

The single mutants of the glutaryl amidase from Pseudomonas SY-77 comprising mutations Asn266 into glutamine, Asn266 into histidine or Asn266 into methionine were combined with different other mutations. Some mutants were purified and the hydrolysis activity towards cephalosporin C acylase was determined. The conversion rate of the different mutants was calculated from an assay with 70 μg enzyme. The mean slope of the conversion of cephalosporin C in the first four hours was determined and normalized towards WT (see FIG. 1). The multiple mutants SY-77^{N266H+Y178H}, SY-77^{N266M+Y178H} and SY-77^{N266Q+Y178H+F375M} appeared to have a higher hydrolysis activity towards cephalosporin C than the wild-type glutaryl amidase (FIG. 3).

### Example 8

### Some Single Mutated Variants of Pseudomonas SY-77 Glutaryl Amidase Carrying a Mutation at Position Asn266 with Increased Hydrolysis Activity Towards Adipyl-7-ADCA

Wild-type (WT) and 15 glutaryl amidase variants mutated at position 266 were produced in E. coli DH10B and purified up to 95% purity. The four glutaryl amidases comprising single mutations N266R, N266I, N266K and N266V could not be produced. The catalytic parameters of the remaining 16 enzymes were determined measuring the initial rate of hydrolysis on a range of substrate concentrations with a fixed amount of enzyme using the Multiprobe II for all pipetting steps. One hundred forty μl 20 mM phosphate buffer pH 7.5 with substrate (0.06-2 mM glutaryl-7-ACA or 0.2-10 mM adipyl-7-ADCA) was preheated at 37° C. Forty μl phosphate buffer containing an appropriate amount of purified enzyme was added, starting the reaction. After ten minutes incubation at 37° C., 40 μl 2.5 M acetate buffer pH 4.5 was added to stop the reaction. Twenty μl 1 mg/ml fluorescamine in acetone was added and the A380 was measured after 60 minutes incubation at room temperature.

Kinetic parameters were obtained by fitting the experimental data from Eadie-Hofstee plots, and the mean and standard deviation of values of at least four independent measurements were calculated. The k_{cat} was calculated using the theoretical molecular weight of the mature enzyme, 75.9 kDa. Variants carrying mutations Asn266 into Met, His or Gln displayed a two- to fifteen-fold improved catalytic efficiency over wild-type according to the hydrolysis of adipyl-7-ADCA, whereas the variants carrying mutation Asn266 into Phe, Trp, Tyr or Cys were equally or 150% as efficient as WT in the hydrolysis of adipyl-7-ADCA. Hence, this example clearly shows that the variants according to the present invention are indeed very effective enzymes for the conversion of adipyl-7-ADCA into 7-ADCA.

### Example 9

### Some Multiple Mutated Variants of Pseudomonas SY-77 Glutaryl Amidase Selected on Adipyl-leucine with Increased Hydrolysis Activity Towards Adipyl-7-ADCA

Two libraries of multiple mutated glutaryl amidase of Pseudomonas SY-77 were constructed. The first one comprised all randomized codons at positions Asn266 and Phe375 as described in Example 3. In the second library, the Tyr178 of all the plasmids of library 1, were replaced by a His. E. coli DH10B cells containing the mutant plasmids were slowly thawed on ice and washed twice with 0.9% NaCl. They were starved by incubation in 0.9% NaCl for two hours at 37° C. and plated onto selective minimal medium plates containing 0.1 mg/ml adipyl-leucine as a sole leucine source. Approximately 5000 viable E. coli DH10B cells containing the mutant plasmids were spread onto each plate. Of each library, a total of 10⁵ transformants was plated. Plates were incubated at 30° C. for at least ten days. Every day, newly appearing colonies were marked.

Colonies were selected on the basis of their date of appearance and size, and streaked again on minimal medium containing adipyl-leucine as sole leucine source to ensure unique colonies were obtained. Single colonies from these second plates were used to inoculate an overnight culture to determine acylase activity and to isolate plasmid DNA for sequence determination. Four colonies with unique sequences appeared to have an improved ratio of adipyl-7-ADCA hydrolysis over glutaryl-7-ACA hydrolysis. These mutants were purified and their catalytic parameters on glutaryl-7-ACA and adipyl-7-ADCA were determined as described in Example 8. Furthermore, the hydrolysis of cephalosporin C was measured as described in Example 2. The hydrolytic properties of these enzymes towards these different substrates are listed in Table 2. It is obvious that all measured mutants have a better catalytic efficiency towards adipyl-7-ADCA, mostly due to a lowered Kₘ. Furthermore, three out of the four mutants tested have an improved hydrolysis rate of cephalosporin C. It is not clear if this activity stems from an improved k_{cat} and/or Kₘ. This example shows, however, that variants of the glutaryl amidase with an improved activity towards adipyl-7-ADCA can be isolated using the described selection method.

### Example 10

### Some Rationally Designed Multiple Mutants of Pseudomonas SY-77 Glutaryl Amidase with Increased Hydrolysis Activity Towards Adipyl-7-ADCA

Combinations were made of mutant Tyr178His of the α-subunit and six mutants of the β-subunit, i.e., F229L, N266H, N266S, M271V+Q291K+T374S, F375H and F375L. All these mutants show an increased activity towards adipyl-7-ADCA. In the six plasmids encoding the genes with mutations in the β-subunit, Tyr178 was replaced by His. The mutant enzymes were purified from E. coli DH10B cells that were transformed with these plasmids as described in Example 1 and their kinetic parameters towards adipyl-7-ADCA were determined as described in Example 8. The hydrolytic properties of the mutant enzymes are listed in Table 3. The k_{cat} towards adipyl-7-ADCA of the combination mutants Tyr178His+Asn266His and Tyr178His+Phe375Leu are significantly higher that that of both “parent” mutants, clearly showing that combining mutations in the gene for the glutaryl amidase can result in mutant enzymes with more improved kinetic parameters.

### Example 11

### Glutaryl Amidase Variants with Increased Hydrolysis Activity Towards Adipyl-7-ADCA Obtained from a Library of Glutaryl Amidase Mutants Randomized at Positions 178, 231, 255, 266 and 375 that was Selected on Adipyl-leucine

A mutant library of glutaryl amidase was constructed as described in Example 4. The region encoding the randomized codons was subsequently amplified using the primers 5′-agggggccgaatactggggcccggattacg and 5′-ctcgaagaccgggccatgga. The product was digested, ligated and transformed as described in Example 4, resulting in a mutant library sized approximately 6×10⁷ in which positions 178, 231, 255, 266 and 375 were randomized.

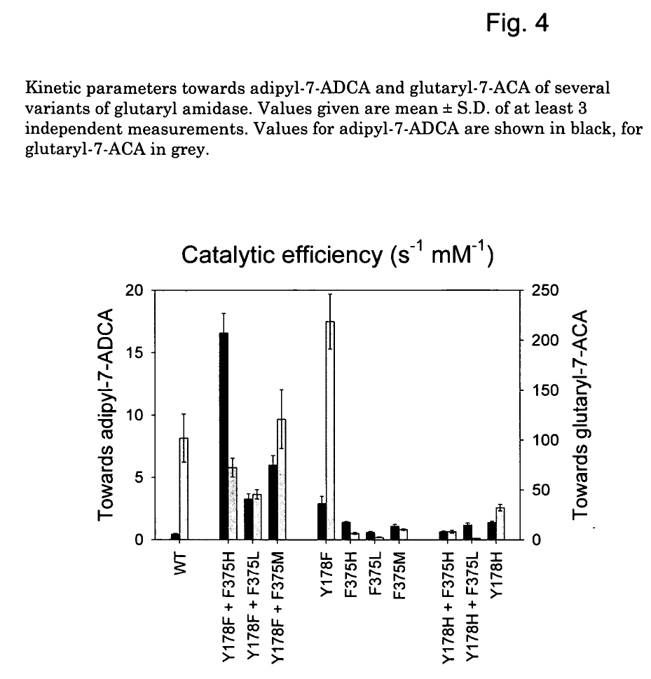

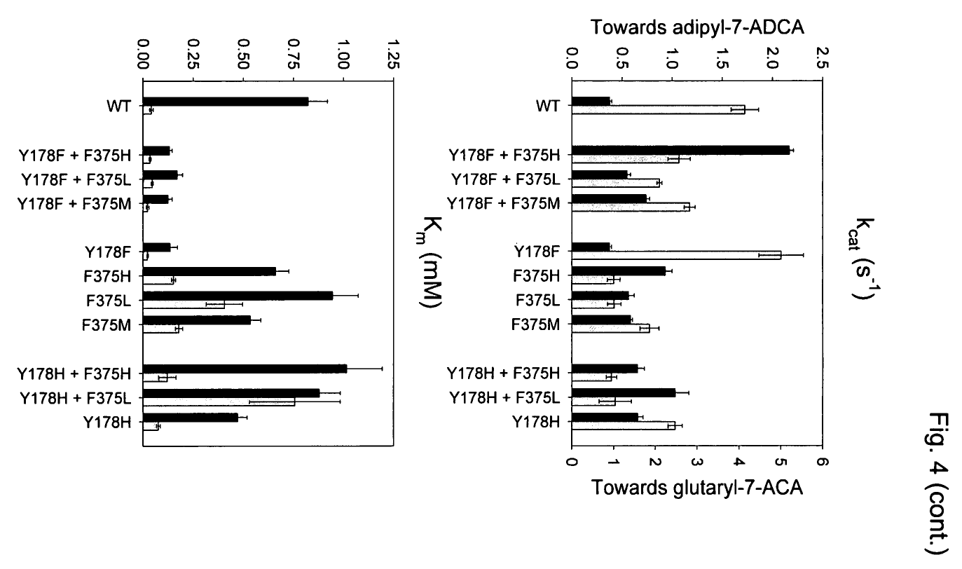

Subsequent sequencing of 20 randomly picked transformants showed that positions 178, 231 and 375 were randomized well, but positions 255 and 266 were randomized to a lesser extent. This mutant library was selected on adipyl-leucine as described in Example 9. Thirty individual colonies were picked from the plates, of which three unique mutants showed an increased activity towards adipyl-7-ADCA: mutants Y178F+F375H; Y178F+F375L; and Y178F+F375M. The three mutant proteins were purified and their catalytic parameters on glutaryl-7-ACA and adipyl-7-ADCA were determined as described in Example 8. The hydrolytic properties of these enzymes towards these different substrates are shown in FIG. 4. It is obvious that all three double mutants show improved kinetic parameters k_{cat}, Kₘ and k_{cat}/kₘ towards adipyl-7-ADCA when compared to the wild-type enzyme. Interestingly, the kinetic parameters are also improved when compared to the values for the respective single mutants, which were made and subsequently purified and characterized in the same manner. The best mutant, Y178F+F375H, showed a 36-fold increase of catalytic efficiency when compared to the wild-type enzyme, due to a six-fold increase of k_{cat} and a six-fold decrease of Kₘ (FIG. 4). The increase in catalytic efficiency and in k_{cat} of this mutant towards adipyl-7-ADCA was more improved than could be calculated from the respective single mutants (36 versus 6.3×3.1=19.4 for catalytic efficiency and 5.8 versus 1.0×2.5=2.5 for k_{cat}), revealing the unique character of this mutant. In contrast, the combination of mutation Y178H with mutation F375H or F375L, as in double mutants Y178H+F375H and Y178H+F375L, does not improve the kinetic parameters more than could be calculated from the respective single mutants. Obviously, the high activity of mutant Y178F+F375H is highly surprising. As a further surprising result, the single mutant Y178F showed improved kinetic parameters towards glutaryl-7-ACA when compared to the wild-type enzyme. To our knowledge, this is the first mutant of glutaryl amidase to show improved activity towards the recognized preferred substrate glutaryl-7-ACA.

TABLE 1

Primers used in the megaprimer method in order to
randomize positions 178, 231, 255 and 266.

| | Position | |
|---|---|---|
| Primer | randomized | Sequence (5′ _> 3′) |
| F1 | 178 | accgcctgatgaacttcctcnnsgtcgcgtcg |
| F2 | | atgtgtggaattgtgagcgg |
| B1 | 231 | gcgcctcgtasnnggtgaagtagtccgtc |
| | | gtcca |
| B2 | 255 + 266 | ccgttgacggtsnnggtgatgcccatccgctg |
| | | gttgaaggcgaasnngatgaccggc |
| B3 | | gattggtgacccgcggca |

TABLE 2
The catalytic parameters of adipyl-leucine selected mutants on
glutaryl-7-ACA and adipyl-7-ADCA. The hydrolytic activity towards
cephalosporin C is given as the relative activity of the wild-type glutaryl
amidase. The values of the basic glutaryl amidases of both
libraries were also measured.

| Sequence | | | | | | | | | |
|---|---|---|---|---|---|---|---|---|---|
| at position | | | glutaryl-7-ACA | | | adipyl-7-ADCA | | | ceph. C |
| 178 | 266 | 375 | k_{cat} | Kₘ | cat/Kₘ | k_{cat} | Kₘ | k_{cat}/Kₘ | rel. act. |
| His | Met | Phe | 0.9 | 0.31 | 2.9 | nd | nd | nd | 1.6 |
| His | Gln | Met | 0.7 | 0.63 | 1.2 | 0.7 | 0.27 | 2.8 | 1.3 |
| Tyr | Gln | Leu | 0.7 | 0.17 | 4.4 | 0.4 | 0.13 | 2.8 | 0.8 |
| His | His | Phe | 2.2 | 0.08 | 26 | 1.0 | 0.18 | 6.2 | 2.6 |
| Tyr | Asn | Phe | 4.6 | 0.06 | 83 | 0.4 | 0.92 | 0.5 | 1 |
| His | Asn | Phe | 5.0 | 0.15 | 40 | 1.2 | 0.8 | 1.5 | 2.7 |

TABLE 3
The kinetic parameters of rationally designed double mutants of glutaryl
amidase towards adipyl-7-ADCA
(Triple = Met271Val + Gln291Lys + Thr374Ser).

| | | | k_{cat}/Kₘ |
|---|---|---|---|
| | k_{cat} (S⁻¹) | Kₘ (mM) | (S⁻¹ mM⁻¹) |
| WT | 0.38 ± 0.02 | 0.8 ± 0.1 | 0.46 ± 0.05 |
| Tyr178His | 0.66 ± 0.05 | 0.47 ± 0.05 | 1.40 ± 0.08 |
| Tyr178His + Phe229Leu | 0.71 ± 0.05 | 1.1 ± 0.2 | 0.7 ± 0.1 |
| Phe299Leu | 0.47 ± 0.02 | 0.52 ± 0.04 | 0.91 ± 0.04 |
| Tyr178His + Asn266His | 0.95 ± 0.04 | 0.19 ± 0.07 | 6 ± 2 |
| Asn266His | 0.55 ± 0.02 | 0.14 ± 0.02 | 3.9 ± 0.3 |
| Tyr178His + Asn266Ser | 0.43 ± 0.06 | 0.9 ± 0.3 | 0.5 ± 0.2 |
| Asn266Ser | 0.34 ± 0.02 | 0.42 ± 0.04 | 0.82 ± 0.05 |
| Tyr178His + Triple | 0.44 ± 0.09 | 0.8 ± 0.2 | 0.6 ± 0.2 |
| Triple | 0.61 ± 0.04 | 0.7 ± 0.1 | 0.9 ± 0.1 |
| Tyr178His + Phe375His | 0.65 ± 0.07 | 1.0 ± 0.2 | 0.65 ± 0.06 |
| Phe375His | 1.16 ± 0.08 | 0.9 ± 0.2 | 1.3 ± 0.1 |
| Tyr178His + Phe375Leu | 1.0 ± 0.1 | 0.9 ± 0.1 | 1.2 ± 0.2 |
| Phe375Leu | 0.67 ± 0.04 | 0.7 ± 0.1 | 0.9 ± 0.1 |

### REFERENCES

1. Yamada H., Y. Ishii, Y. Noguchi, T. Miura, T. Mori and Y. Saito (1996) Protein engineering of a cephalosporin C acylase. Ann. N.Y. Acad. Sci. 799, 74-81.
2. Ishii Y., Y. Saito, T. Fujimura, H. Sasaki, Y. Noguchi, H. Yamada, M. Niwa and K. Shimomura (1995) High-level production, chemical modification and site-directed mutagenesis of a cephalosporin C acylase from Pseudomonas strain N176. Eur. J Biochem. 230, 773-778.
3. Saito Y., T. Fujimura, Y. Ishii, Y. Noguchi, T. Miura, M. Niwa and K. Shimomura (1996) Oxidative modification of a cephalosporin C acylase from Pseudomonas strain N176 and site-directed mutagenesis of the gene. Appl. Environ. Microbiol. 62, 2919-2925.
4. Saito Y., Y. Ishii, T. Fujimura, H. Sasaki, Y. Noguchi, H. Yamada, M. Niwa and K. Shimomura (1996) Protein engineering of a cephalosporin C acylase from Pseudomonas strain N176. Ann. N.Y. Acad. Sci. 782, 226-240.
5. Fritz-Wolf K., K. P. Koller, G. Lange, A. Liesum, K. Sauber, H. Schreuder, W. Aretz and W. Kabsch (2002) Protein Sci. 11, 92-103.
6. Sio C. F., A. M. Riemens, J. M. van der Laan, R. M. D. Verhaert and W. J. Quax (2002) Directed evolution of a glutaryl acylase into an adipyl acylase. Eur. J. Biochem. 269, 4495-4504.
7. Elander R. P. (2003) Appl. Microbiol. Biotechnol. 61, 385-392.
8. Oh B., M. Kim, J. Yoon, K. Chung, Y. Shin, D. Lee and Y. Kim (2003) Deacylation activity of cephalosporin acylase to cephalosporin C is improved by changing the side-chain conformations of active-site residues. Biochem. Biophys. Res. Commun. 10; 310(1):19-27.
9. Otten L. G., C. F. Sio, J. Vrielink, R. H. Cool and W. J. Quax (2002) J. Biol. Chem. 277 (44), 42121-42127.
10. Ishii Y., Y. Saito, T. Fujimura, T. Isogai, H. Kojo, M. Yamashita, M. Niwa and M. Kohsaka (1994) A Novel 7-β-(4-carboxybutanamido)-cephalosporanic acid acylase isolated from Pseudomonas strain C427 and its high-level production in Escherichia coli. Journal of Fermentation and Bioengineering, 77, 591-597.
11. Li Y., J. Chen, W. Jiang, X. Mao, G. Zhao and E. Wang (1999) In vivo post-translational processing and subunit reconstitution of cephalosporin acylase from Pseudomonas sp. 130. Eur. J. Biochem. 262, 713-719.
12. Li Y., W. Jiang, Y. Yang, G. Zhao and E. Wang (1998) Overproduction and purification of glutaryl 7-amino cephalosporanic acid acylase. Protein Expr. Purif. 12, 233-238.
13. Ichikawa S., Y. Murai, S. Yamamoto, Y. Shibuya, T. Fujii, K. Komatsu and R. Kodaira (1981) The isolation and properties of Pseudomonas mutants with an enhanced productivity of 7-β-(4-carboxybutanamido)-cephalosporanic acid acylase. Agric. Biol. Chem. 45, 2225-2229.
14. Matsuda A. and K. I. Komatsu (1985) Molecular cloning and structure of the gene for 7-β-(4-carboxybutanamido)-cephalosporanic acid acylase from a Pseudomonas strain. J. Bacteriol. 163, 1222-1228.
15. Kim D. W., S. M. Kang and K. H. Yoon (1999) Isolation of novel Pseudomonas diminuta KAC-1 strain producing glutaryl 7-aminocephalosporanic acid acylase. J. Microbiol. 37, 200-205.
16. Kim Y., K. Yoon, Y. Khang, S. Turley and W. G. Hol (2000) The 2.0 Å crystal structure of cephalosporin acylase. Structure 8, 1059-1068.
17. Shibuya Y., K. Matsumoto and T. Fujii (1981) Isolation and properties of 7-β-(4-carboxybutanamido)-cephalosporanic acid acylase-producing bacteria. Agric. Biol. Chem. 45, 1561-1567.
18. Verhaert et al. (1997) Appl. Environ. Microbiol. 1997 September; 63(9):3412-8.
19. Biokatalysatoren und Enzymtechnologie, K. Buchholz and V. Kasche, VCH Verlag, Weinheim, 1st edition 1997.
20. Cao L., L. Van Langen and R. A. Sheldon (2003) Immobilized enzymes: carrier-bound or carrier-free? Curr. Op. Biotechnol. 14, 387-394.
21. Bruggink A., E. C. Roos and E. de Vroom (1998) Organic Process Res. Dev. 2, 128-133.
22. Ausubel F. et al, Current Protocols in Molecular Biology, Greene Publishing Associates and Wiley Interscience, 1994-1998 and Supplements.
23. Landt O., H. P. Grunert and U. Hahn (1990) Gene 1990, 96 125-128.
24. Reyes F., M. J. Martinez and J. Soliveri (1989) Determination of cephalosporin-C amidohydrolase activity with fluorescamine. J. Pharm. Pharmacol. 41(2):136-7.

1. An isolated or single mutated glutaryl amidase from Pseudomonas SY-77, or a functional part, derivative or analogue thereof, having an amino acid at position 266 other than asparagine, histidine or serine.

2. The isolated or single mutated glutaryl amidase of claim 1, having a glutamine, methionine or tryptophan at position 266.

3. The isolated or single mutated glutaryl amidase of claim 2, having a glutamine or methionine at position 266.

4. The isolated or single mutated glutaryl amidase of claim 3, having a glutamine at position 266.

5. An isolated or multiple mutated glutaryl amidase from Pseudomonas SY-77, or a functional part, derivative or analogue thereof, having an amino acid other than its native amino acid at position Asn266, and having an amino acid other than its native amino acid at one or more positions Leu177, Tyr178, Val179, His221, Leu222, Phe229, Tyr231, Gln248, Arg255, Phe256, Thr267, Val268, Met271, Gln291, Met347, Tyr351, Thr374, Phe375 and/or Asn442.

6. The isolated or multiple mutated glutaryl amidase of claim 5, having an amino acid other than its native amino acid at one or more positions Tyr178, Leu222, Tyr231, Gln248, Arg255, Val268, Tyr351 and/or Phe375.

7. The isolated or multiple mutated glutaryl amidase of claim 5, having an amino acid other than its native amino acid at one or more of the following positions: Tyr178, Tyr231, Arg255, Phe375, Tyr178+Tyr231, Tyr178+Arg255, Tyr178+Phe375, Tyr231+Arg255, Tyr231+Phe375, Tyr231+Arg255+Phe375, Arg255+Phe375, Tyr178+Tyr231+Arg255, Tyr178+Tyr231+Phe375, Tyr178+Arg255+Phe375, or Tyr178+Tyr231+Arg255+Phe375.

8. The isolated or multiple mutated glutaryl amidase of claim 5, having a histidine, glutamine, methionine or tryptophan at position 266.

9. The isolated or multiple mutated glutaryl amidase of claim 8, having a histidine, glutamine or methionine at position 266.

10. The isolated or multiple mutated glutaryl amidase of claim 9, having a glutamine at position 266.

11. An isolated polynucleotide encoding the glutaryl amidase of claim 1.

12. A nucleic acid vector comprising the polynucleotide of claim 11.

13. A host cell comprising a polynucleotide of claim 11, which polynucleotide is operably linked to a regulatory control sequence which allows for direct expression of the variant of a glutaryl amidase in said host cell.

14. A method for producing the glutaryl amidase, which method comprises a culturing:
the host cell of claim 13 under suitable conditions which allows for expression of the glutaryl amidase in said host cell.

15. A method for preparing 7-aminocephalosporanic acid from cephalosporin C, which method comprises:
reacting cephalosporin C with an isolated, single mutated or multiple mutated glutaryl amidase from Pseudomonas SY-77, or a functional part, derivative or analogue thereof, having an amino acid at position 266 other than asparagine under suitable conditions such that the glutaryl amidase cleaves the cephalosporin C directly to form the 7-aminocephalosporanic acid.

16. A method for preparing 7-aminocephalosporanic acid from cephalosporin C, which method comprises reacting:
cephalosporin C with a variant of the glutaryl amidase of claim 1 under suitable conditions such that the glutaryl amidase cleaves the cephalosporin C directly to form the 7-aminocephalosporanic acid.

17. A method for preparing 7-aminocephalosporanic acid from cephalosporin C, which method comprises:
reacting cephalosporin C with an isolated, single or multiple mutated glutaryl amidase from Pseudomonas SY-77, or a functional part, derivative or analogue thereof, having an amino acid other than its native amino acid at position Tyr178, under suitable conditions such that the glutaryl amidase cleaves the cephalosporin C directly to form the 7-aminocephalosporanic acid.

18. The method according to claim 17, wherein the glutaryl amidase has a histidine at position Tyr178.

19. The method according to claim 16, wherein the glutaryl amidase is a single mutated glutaryl amidase.

20. A method for preparing aminodesacetoxycephalosporanic acid, which method comprises:
reacting adipyl-7-ADCA with an isolated or single mutated glutaryl amidase from Pseudomonas SY-77 or a functional part, derivative or analogue thereof, having an amino acid other than its native amino acid at position Asn266, under suitable conditions such that the glutaryl amidase cleaves adipyl-7-ADCA to form the aminodesacetoxycephalosporanic acid.

21. A method for developing a glutaryl amidase with cephalosporin C hydrolysis activity, said method comprising the steps of:
a) applying a mutagenesis technique in order to create a large library of mutants of the glutaryl amidase of the present invention that carries a mutation in one or more positions; and subsequently
b) applying a selection procedure comprising growth of proper host cells that express variants of said glutaryl amidase on solid medium containing a selective, aminoadipyl-containing compound and identification of variants with the desired enzymatic activity.

22. A method for preparing 7-ACA based antibiotics, said method comprising:
incubating 7-ACA in the presence of the glutaryl amidase of claim 1 with alkyl side chains under suitable synthesizing conditions.

23. An isolated or multiple mutated glutaryl amidase from Pseudomonas SY-77, or a functional part, derivative or analogue thereof, having a phenylalanine at position 178 and a histidine at position 375.

24. The isolated or multiple mutated glutaryl amidase from Pseudomonas SY-77 of claim 5, or a functional part, derivative or analogue thereof, having a phenylalanine at position 178 and a histidine at position 375.

25. A polynucleotide encoding the glutaryl amidase of claim 23.

26. A nucleic acid vector comprising the polynucleotide of claim 25.

27. A host cell comprising the polynucleotide of claim 26, which polynucleotide is operably linked to a regulatory control sequence which allows for direct expression of the variant of a glutaryl amidase in said host cell.

28. A method for producing isolated or multiple mutated glutaryl amidase from Pseudomonas SY-77, which method comprises:
culturing a host cell according to claim 27 under suitable conditions which allow for expression of the glutaryl amidase in said host cell.

29. A method for preparing 7-aminodesacetoxycephalosporanic acid, which method comprises:
reacting adipyl-7-ADCA with a mutated glutaryl amidase from Pseudomonas SY-77 according to claim 23, or a functional part, derivative or analogue thereof, under suitable conditions such that the glutaryl amidase cleaves adipyl-7-ADCA to form the 7-aminodesacetoxycephalosporanic acid.
